(19) United States
(12) Patent Application Publication
(10) Pub. No.: US 2005/0133041 A1
(43) Pub. Date: Jun. 23, 2005

(54) Pre-ejaculation condom

(76) Inventor: Michael Tune, Colorado Springs, CO (US)

Correspondence Address: John P. Luther, Esq.;Newman & Newman, LLP, Suite 610, 505 Fifth Avenue South, Seattle, WA, 98104, US

(21) Appl. No.: 10/743,925

(22) Filed: Dec. 23, 2003

(51) Int. Cl. A61F 6/02

(52) U.S. Cl. 128/842

(57) ABSTRACT

The present invention provides a contraceptive prophylactic condom to prevent the transmission of seminal fluid during sexual intercourse and/or fellatio comprising a closed end and an open end opposite the closed end, an intermediary portion between said ends generally in a frusto-conical shape and/or mimicking the size and shape of a glans penis of the male sex organ, wherein the open end forms a circumferential leak proof enclosure of its periphery about the glans penis portion at a position above the sulcus, and wherein said condom contains a securing means on its inner surface effective to maintain a non-movable position on the glans penis during intercourse or fellatio.

### FIELD OF THE INVENTION

The present invention relates generally to condoms, and more particularly to condoms used for capturing pre-ejaculation fluid, or pre-cum, and especially such condoms for use during fellatio.

### BACKGROUND OF THE INVENTION

Many people have long used condoms during sexual intercourse for different reasons and in recent times for the most part, to avoid the transmission of sexually transmitted diseases (“STD's). Many different and varied types of condoms are known, of which they are usually of the type applied to a male penis in the form of a flexible material placed over the penis or the vaginal canal of a woman. Male condoms are usually formed from impermeable material, electrometric in nature, and are generally tubular with an open end and a closed end. Such condoms are stretched to fit tightly around a penis such that many instances frictional movement between the penis and the condom material is minimized, or preferably eliminated. In many improved condoms it has been sought to minimize the thickness of the condom material for the wearer's heightened sensory benefit.

An example of an improved version of condom is described in WO-1989002256 to Brook, which is formed of impermeable material and tubular in shape with an open end and a closed end and electrometric in nature with respect to the portion adjacent the open end. This condom further comprises an enlarged portion remote from the open and which is said to be sufficiently enlarged to permit relative movement between the enlarged portion of the condom and a penis portion fitted with the condom. Such an arrangement is aid to provide for sliding friction to be felt by the wearer, and which is absent in conventional condom.

A further example, as disclosed in WO 9014048 to Friedman, which is apparently designed more for sensory intensity than for protection against STD's, comprises an unrolled condom encompassing solely the head of a penis having a reservoir to collect semen and an adhesive means to securely fasten the condom to the head of the penis. The device further contains an angled condom opening to solely encompass the head of the penis, and an internal retaining means within the center of the flange lip which in use becomes integrally secured under the lip on the head of a penis to allow the rest of the shaft to be exposed. This design is said to increase sensitivity during intercourse to form a more secure fastening arrangement between the penis and condom. Additionally, a retaining means of the condom is angularly designed to relate functionally and encompass the angle of the lip of the penis head and the angle of the penis shaft. Adhesive is applied throughout the top and bottom portions of the condom to securely fasten the condom to the head of the penis.

Other examples of condoms and improved versions are myriad. For example, WO-1994028833 to Strozer discusses and improved male condom which is loose fitting and held in place by a continuous elastic band or inelastic band of Velcro, to provide a more easily applied and removed condom with increased sensation for the wearer. This variation contains a seal at the base of the penis to protect from disease and pregnancy. If the base is suitable tight, it is also said to benefit from prolonged erections. WO2001047446 in providing yet another example, discusses a condom set wherein a separate thin membrane pocket is filled with local anesthetic, and sealed in place on a selected condom penis contact location, which is activated before use by rupturing the thin membrane pocket to apply local anesthetic to the predetermined penis location in use, and which is effective, no doubt, in preventing premature ejaculation. In EP 324557, there is described a condom constructed of a certain configuration applied with medical grade adhesive effective to from a leak-free seal with the tip (glans section) of the penis, and said to eliminate the need for a roll down hood, and thereby providing for increased user pleasure with reduced risk of an unwanted pregnancy and STD's infection. Further examples include condoms with spiral crisscross ribbing as disclosed in Published application No. 20010023696; condoms having an anatomically correct shape in Published application No. 200020189619; condoms with desensitizer lubricants in Published application No. 2002010344; polyisoprene condoms disclosed in Published application No. 20030161975; colored and/or multi-colored high-protective multi-layered polymer condoms of Published application No. 20030124354; aloe vera impregnated condoms of Published application No. 20020114825; and caps to be used as protective coverings on rolled condoms, and their use with condoms for added protection in U.S. Pat. No. 4,726,359; an alarm clock prophylactic dispensing system, as disclosed in Published application No. 20030160058; and condoms for dispensing a flavored composition into the body orifice to be penetrated, as disclosed in U.S. Pat. No. 4,919,149.

Despite the many and varied types of condoms, and specialty condoms, as shown by only a sampling of the above examples, there have not been many provided or designed for use in oral-genital sexual contact, which as many will acknowledge is engaged in frequently.

One of the few examples of a condom for oral-genital use is described in EP 600858 to Burr, which provides a condom-like prophylactic designed to permit oral-sex while preventing STD contraction. Such a condom is adapted to be worn in the mouth of the person practicing oral sex and is fabricated as tubular member with a closed end, and formed of thin, plaint impervious material. The open end of the tubular member is joined to a pair of flanges which extend radially outward, and which can be curved out of a plane transverse to the axis of the tubular member to form a channel of the general configuration of lips and mouth to generally conform to the wearer. The tubular member extends into the mouth of the wearer, with a penis inserted into the tubular member in the performance of fellatio all the while preventing direct contact with lips, mouth or the tongue of the wearer. In another embodiment, the tubular member can extend outwardly to contain the extended tongue of the wearer in the performance of cunnilingus, again without direct contact with the vulva. Such a device, however, is cumbersome to the wearer, and difficult to manufacture to fit the endless differently sized mouth features of the general public, not to mention its adverse Halloween mask-like effect on any romantic mood.

Another example of a condom for oral sexual usage is described in WOA 19890001 in the form of a tubular portion for location in a mouth cavity over the tongue of the wearer and a pocket portion for receiving the tongue of the wearer. This condom, which is designed to fit over the human mouth and lips, however suffers from the same shortcomings of the above-mentioned fellatio device.

Some additional examples of condoms include U.S. Pat. No. 5,421,350 to Friedman which discloses a condom adhesive means which is specifically designed for retaining a condom which is small to increase male sensation and heightened enjoyment. This condom is described as a flexibly cylindrical member which when rolled outwardly upon itself releases adhesive to provide a secure attachment to the head of a penis, an angled condom opening to solely encompass only the head of the penis, and an internal retaining means within the center of a flange which becomes integrally secured under the lip on the head of the penis, thereby allowing the rest of the penis to be exposed to increase sensitivity during intercourse. The retaining means in this example is designed as a first angle that parallels an angle of the penis head and a second angle that parallels an angle of the penis shaft and which can be integrally molded within a flange portion of the open end of the condom.

U.S. Pat. No. 6,298,853 describes facilitating the securement of a condom of abbreviated length to the glans penis area via a pair of bilaterally symmetric areas provided for the lobes of the glans penis. This condom contains, among other things, a viscous layer on the interior surface which is said to enhance suction characteristics and increase cohesion between condom and skin contract, a reservoir for seminal fluid, and a reversible margin proximate to the open rear end of the condom which is said to fit into the sulcus portion of the penis, inclusive of a circumferential adhesive layer to obtain a seal against fluid transmission.

U.S. Pat. No. 4,869,269 to Sharkon provides a contraceptive, prophylactic condom formed to configure to the shape of the tip, or glans penis, of the male sex organ. This condom device is described as having a relatively narrow elongated portion on one end for collecting seminal fluid and a wider, generally frusto-conical portion which fits over and adheres to the glans penis, including an indented portion corresponding to an indented portion in the underside portion of the glans penis anatomy to hold the condom in place and form a leak-free seal with the glans penis. A pressure-sensitive medical grade adhesive is also employed to hold the condom at its position. The condom is characterized as only covering the glans penis and not the “corona”, or ridge surrounding the glans penis, or the “frenulum” or underside of the penis where the glans meets the forskin, which are said to be the most sensitive part of the male sex organ.

U.S. Pat. No. 6,298,852 to Manning teaches an extension condom inclusive of a sponge said to simultaneously extend the size of the penis while preventing the transfer of micro-organisms between sex partners.

Yet another example of adhesive use in condoms is seen in U.S. Pat. No. 3,677,225 to Czirely. This reference discloses a contraceptive device to be adhesively applied to the tip of the male sex organ which is an elongated seminal receptacle having an open end and a closed end with an interior adhesive layer situated on the interior surface of the receptacle at the open end extending annularly about an entire cross-sectional portion of the device. A protective webbing is also removably attached to the exterior of the receptacle for handling without damage to the adhesive layer.

U.S. Pat. No. 6,035,854 teaches another abbreviated condom provided with a molded, contoured or thickened wedge portion to fit snuggly with the cleft portion of the glans, such that a continuous circumferential seal is formed about the glans penis. An expansible seminal reservoir is also provided. As an option, the shaft of the penis, as well as a rear portion of the glans penis opposed to the cleft can be felt unsheathed to allow greater sensitivity. An adhesive backing with a tab is also employed to permit device location prior to removal of the backing to ensure correct positioning of the condom device and an effective circumferential seal with the adhesive. A wedge shaped structure fitting in the glans is utilized to obtain an anatomically accurate interior annular surface, and to which bears an adhesive layer, or an elastic band is used in the case of the entire corona being encompassed.

U.S. patent No. 20020139373 teaches further condom variations such as the provision of a lollipop-shaped condom inclusive of a stick-resembling portion of smaller circumference than the shaft of a male member, and to which it is fitted and an “installation ring” necessary to install the condom having a small circumference area.

In U.S. Pat. No. 6,089,231, there is disclosed a contoured micro-condom including a thin impervious sheath in the general shape of the glans penis head contour, which employs a thicker elastic band around its open end. The band is described as being positioned securely and is contained within an aperture within the retainer frame of the application/package and wherein the aperture has sufficient width to accommodate the diameter of a normal erect glans penis head. A narrow elastic band portion is described as shaped and sized especially for placement into the coronal sulcus of the glans. As further explained, the more exact non-linear tapering of the radii to approximate the curves of a normal glans penis is said to enhance the fit of the frusto-conical-shaped micro-condom.

As can be seen, there exists dearth of commercially available condoms designed to facilitate or accommodate oral-genital sex, especially such condoms which are comfortable for the wearer to provide maximum sensual pleasure, as well as comfort and non-hindrance for the person performing fellatio. Additionally, condoms known in the prior art are not known or designed for maximized sensual pleasure while containing pre-ejaculation fluid (pre-cum), which some performers of fellatio may find undesirable, or even offensive, preferring instead to finalize ejaculation through conventional intercourse. Such is a major failing of the prior art, especially since many individuals participate in fellatio with the intent of arousing and stimulating the receiver while simultaneously sustaining sensual pleasure, without the desire to receive or otherwise be exposed to seminal fluid orally, be it pre-cum or ejaculate. As is commonly known, many persons of both sexes desire to participate in oral sex, but oftentimes are hesistant as to the thought of receiving or otherwise being contact with seminal fluid. Such people have effectively been denied safely and pleasurably benefitting themselves and sex partners due to the absence of suitably designed condoms for oral sex.

As also shown, many condoms currently available on the market are not designed nor in any way directed toward oral use, either impliedly or inherently, as lubricants, and adhesives typically provided in condom packages are associated with odors and tastes that are many times offensive and in many instances adverse to fellatio.

### SUMMARY OF THE INVENTION

In accordance with the above noted shortcomings of conventional condoms, the present invention provides a condom specifically designed for fellatio and which provides the maximun amount of sensual pleasure to the fellatee safely and in a manner wholly non-offensive to even the most fastidious individuals who do not wish to receive or be in contact with pre-ejaculation fluid or ejaculate, but yet wish to pleasurably benefit their sexual partners and themselves. Therefore, the present invention provides a condom suitable for oral-genital use comprising a seminal receptacle of impervious material having an open end and a closed end, optionally comprising an enlarged receptacle at the open end to contain all, or substantially all, pre-ejaculate, or ejaculate, provided from a fellatee, and which extends from the closed end to the open end thereby forming an annular circumferential fit around the glans penis, wherein the circumferential periphery thereof extends over the glans penis to a point thereon in which the sulcus is not covered and optionally not covering the frenulum as well, and further wherein said condom is firmly held in place by a positioning means which can be situated on the inner orifice of the condom to secure the condom to the glans penis in a seminal fluid leak-free nature. Another optional feature is to provide an elastometric band integral with the open end of the condom, preferably its periphery, which can be used in conjunction with an adhesive means or other positioning means or alone, and which extends circumferentially around a portion of the glans penis to hold it in place during use.

The presently inventive condom is generally of frusto-conieal design, but can be of a shape, adjacent to and extending outwardly from an optional seminal receptacle, corresponding approximately in size and shape to the anatomy of the glans penis of the male sex organ.

The invention will be better understood and its numerous objects and advantages will become more apparent to those skilled in the art by reference to the following drawings in conjunction with accompanying specification.

### BRIEF DESCRIPTION OF THE DRAWINGS

FIG. 1 is elevational perspective view illustrating a conventional condom as applied to a penis.

FIG. 2 is an elevational perspective view illustrating a condom of the present invention as applied to a penis.

FIG. 3 is an elevational perspective transparent view illustrating a further embodiment of a condom of the present invention as applied to a penis.

FIG. 4 is an elevational perspective transparent view illustrating further embodiments of a condom of the present condom as applied to a penis.

FIG. 5 is a perspective view of two embodiments of a condom of the present invention as they would appear by themselves.

### DETAIL DESCRIPTION OF THE PREFERRED EMBODIMENTS

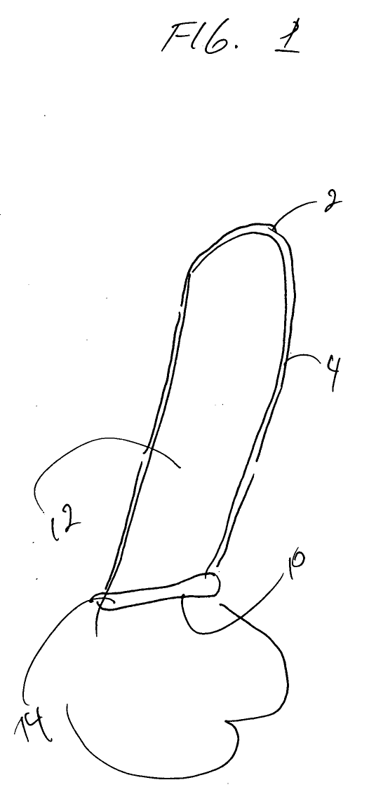

Turning now to the drawings, in FIG. 1, there is shown in elevational perspective view, a conventional condom as applied to a penis. As shown, there is included a closed portion 2 defining one end of the condom 4 covering and generally contoured to the glans penis portion of a penis, and with a tubular portion annularly extending down and covering the majority of the shaft of the penis as depicted in erection. The open end 10 of the condom 4 defines an annular circumference, the periphery of which completely surrounds a portion of the bottom shaft end of the penis 12 opposite the glans penis portion. The condom is shown as also equipped with an elastomeric retaining ring 14 which forms a tight, firmly grasping restricting fit over the circumferential base area of the penis to hold the condom in place and to ensure a tightly fitting barrier against the flow of seminal fluid emanating from the penis.

Such a traditional condom is typically manufactured from non-porous materials, elestomeric in nature, such as, for example, latex rubber, polyurethane and lambskin, and which prevents the transmission of semen during sexual intercourse both in the vaginal passage and oral cavity when so disposed.

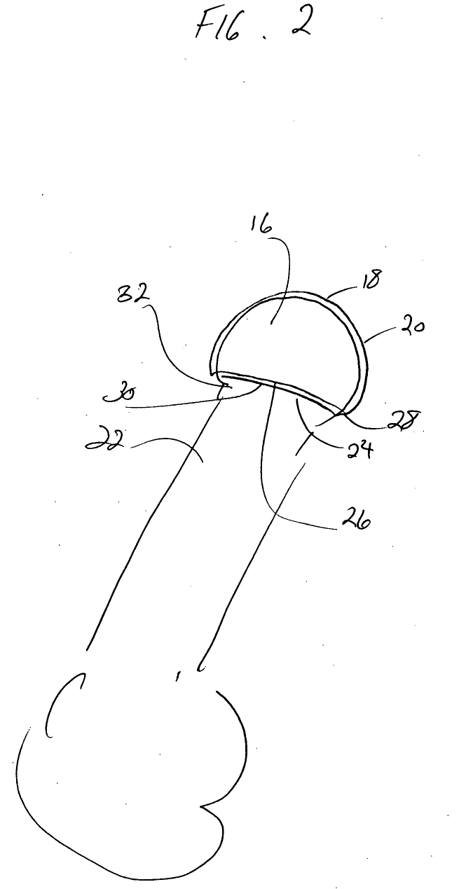

In FIG. 2, there is depicted an elevational perspective view of a preferred embodiment of the present inventive condom in its application to an erect penis. As shown inventive condom 16 comprises a closed end 18 from which a tube-shaped main body 20 extends to completely encloses a majority of the glans penis portion of a penis 22. At the base 24 of the main body 20 is the circular opening 26. The main body 20, depending upon personal preference, can be constructed from any conventional or non-conventional porous, non-porous, solid or absorbent materials, or a combination thereof, with examples including rubber, latex, polyurethane, polyisoprene animal skin, epidermal tissue, plastics, plastic composites, cotton, synthetic cloth, tissue, and synthetic skins and sponges.

An open end 28 of condom 16, opposite from closed end 18 at the opposite end of main body 20, completely surrounds, encloses and encompasses a portion of the glans penis with the circumferential periphery 30 thereof extending around the glans penis just short of the sulcus 32.

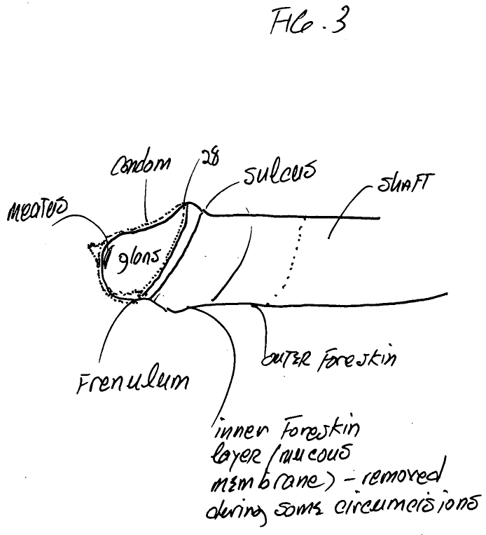

In another preferred embodiment, as shown in FIG. 3 in an elevational perspective transparent view, open end 28 will have a circumferential periphery extending around the glans penis without encompassing or enclosing both a frenulum, or a portion thereof, and the sulcus 32, thereby exposing all or a portion of the frenulum and sulcus and the great majority of the penis to oral contact during fellatio for maximum sensory pleasure to a fellatee while still containing and/or preventing transmission of pre-ejaculate and ejaculate.

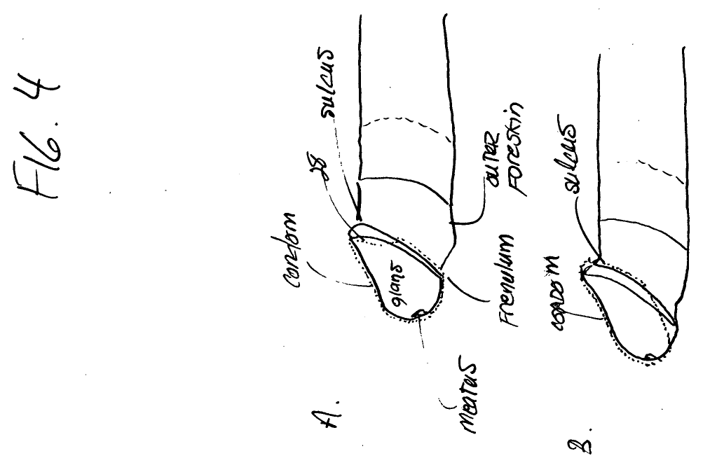

Turning now to FIG. 4, there is shown in a transparent elevational perspective view additional embodiments of the present invention. In these embodiments, the periphery of open end 28 is fitted with an integral retaining means, such as a elastomeric band, to extend around the glans penis thereby covering a portion of the corona area and to fit snugly around and under at least a portion of the lip on the head of the penis and wherein said portion is juxtaposed to a portion of the sulcus. This embodiment is thought to provide for additional positional securement to the glans penis during use, whether it be sexual intercourse or fellatio. Again, an adhesive means may also be used in conjunction with the integral, annual retaining ring, or any other securing means contemplated.

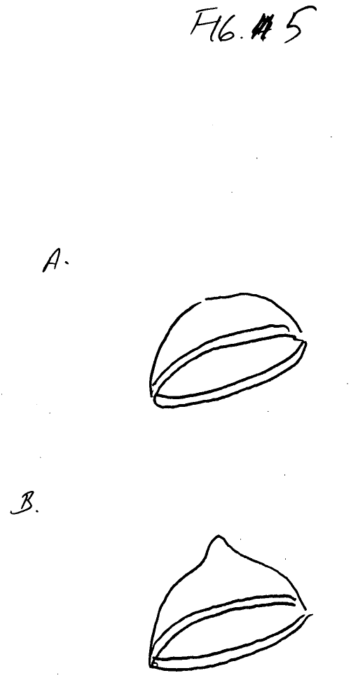

FIG. 5 shows in perspective view embodiments of the present invention as they would appear before deployment, with an inventive condom being equipped with (FIG. 4B) an expandable reservoir means to capture and contain seminal fluid, and an embodiment (FIG. 4A) without being equipped with such means.

As is known, the most important components of the physical erogenous stimulation of the penis during foreplay, especially fellatio and intercourse, are the sensations from the foreskin, frenulum/frenor band and the glans penis. As each of these structures have their own feeling contributing in its own way to a recipient's total experience of lovemaking, it is most desirable to have these areas available for contact during fellatio, all of which is made possible by the present invention.

The periphery 30 of open end 28 may optionally contain an integral elastomeric circumferential portion in the form of an elastomeric band to function as an annular retaining ring disposed around a portion of the glans penis to snugly and securely deploy the condom on the penis effective against the transmission of fluids.

Alternatively, alone or in combination with an annular retaining ring, an adhesive means may be employed to maintain the deployed condom in place about the glans penis during fellatio or intercourse, as the case may be. Such adhesives are well known, and may posses one or more properties, such as being an effective viralcidal bacterial, spermicidal, myocardial, and hypoallergenic agent and may be a medical grade adhesive, pressure sensitive adhesive or a semi-adhesive for easy removal.

Additionally, in accordance with the inventive condom, closed end 18 may include an expandible reservoir means located and integral with closed end 22 to enclose the meatus portion of the erect penis to contain all, or substantially all, of the seminal fluid or ejaculate produced by the fellatee.

It is contemplated that the inventive condoms be available in array of colors, or any combination of colors. The inventive condom may also be provided in any one of several flavors. Flavoring, as well as one or more of the properties disclosed above, may be imparted to the condom by any conventional means, including coatings, powdered sugar coatings, syrup coatings and like, and may be composed of material fit for human consumption to provide edible condoms.

It is further contemplated in this invention that the inventive condoms be employed as a delivery vehicle for vitamins, nutrients and analgesics, such as, for example, aspirin, to combat the proverbial headache one's partner and would-be-fellatio provider sometimes or oftentimes is allegedly afflicted with. The oral condom of the invention provides an ideal delivery vehicle for such substances during performance of fellatio, and may be disposed throughout or otherwise imparted to, the condom by any conventional means such as the application of a coating, such as a powder coating, or incorporated into a dissolvable sugar coating. Other oral-condom deliverable substances contemplated for use in this invention include any of the several known neutraceuticals, such as ginseng and the like, and any other orally deliverable health beneficial substance, the range and scope of which is limited only by the imagination.

While the embodiments described in the foregoing are at present considered to be preferred, such are for illustrative purposes only and are not intended to limit the scope or claims of the invention in any way. It is to be understood that various modifications and improvements may be made therein without departing from the invention as indicated in the appended claims and all changes that come within the meaning and range of equivalency of the claims are intended to be embraced therein.

1. A contraceptive prophylactic condom effective to prevent the transmission of seminal fluid during sexual intercourse and/or fellatio, comprising a closed end and an open end opposite the closed end, an intermediary portion between said ends generally in a frusto-conical shape and/or mimicking the size and shape of a glans penis of the male sex organ, wherein the open end forms a circumferential leak proof enclosure of its perphery about the glans penis portion at a position above the sulcus, and wherein said condom contains a securing means effective to maintain a non-movable position on the glans penis during intercourse or fellatio.

2. The condom of claim 1, further comprising a seminal fluid receiving receptacle on the closed end.

3. The condom of claim 1, which is composed of an impervious material.

4. The condom of claim 1, which is composed of a colored material.

5. The condom of claim 1, which further comprises a flavoring means.

6. The condom of claim 1, which further comprises means for delivery of a substance selected from aspirin or analgesics, vitamins, neutraceuticals, antacid, breath enhancers, nicotine, caffeine and stimulants and/or any combination thereof.

7. The condom of claim 1, wherein said securing means is an adhesive means.

8. The condom of claim 7, wherein the adhesive means is a medical adhesive.

9. The condom of claim 7, wherein the adhesive means is a pressure sensitive adhesive.

10. The condom of claim 7, wherein the adhesive means is viralcidal.

11. The compound of claim 7, wherein the adhesive means is bactericidal.

12. The compound of claim 7, wherein the adhesive means is spermicidal.

13. The compound of claim 7, wherein the adhesive means is a an organic substance.

14. The compound of claim 7, wherein the adhesive means is mycocidal or effective against myocardial infarction.

15. The compound of claim 7, wherein the adhesive means is hypoallergenic.

16. The compound of claim 1, wherein said condom is manufactured from the group consisting of rubber, latex, polyurethane, polyisoprene, plastic, a composite plastic composition, an animal product, cloth and any combination thereof.

17. The condom of claim 7, further comprising an elastomeric band around the periphery of the closed end and effective to form a leak-proof annular seal around the circumference of a portion of the glans penis above the sulcus of a male sex organ.

18. The condom of claim 1, wherein said securing means comprised an elastomeric band around the periphery of the closed end and effective to form a circumference of a portion of the glans penis above the sulcus of a male sex organ.

19. The condom of claim 1, wherein said condom is made of an absorbent material.

20. A method of conducting business comprising the manufacture and/or sale and/or distribution of a condom of claims 1-20.
